(19) United States
(12) Patent Application Publication
(10) Pub. No.: US 2018/0280615 A1
(43) Pub. Date: Oct. 4, 2018

(54) ELECTROLYTE ADJUSTMENT INFUSION SYSTEM AND METHOD

(71) Applicant: SEIRATHERM GMBH, Herzogenaurach (DE)

(72) Inventors: Matthias ROTH, Giggenhausen (DE); Thomas REICHTHALHAMMER, Polling (DE)

(73) Assignee: SEIRATHERM GMBH, Herzogenaurach (DE)

(21) Appl. No.: 15/764,670

(22) PCT Filed: Sep. 29, 2016
(86) PCT No.: PCT/EP2016/073245

§ 371 Date: Mar. 29, 2018

(30) Foreign Application Priority Data
Sep. 30, 2015 (EP) EP15187792.5

(51) Int. Cl. A61M 5/168; A61M 5/14; A61M 5/44

(52) U.S. Cl. CPC A61M 5/16827; A61M 5/1407; A61M 5/445; A61M 2202/0413; A61M 2205/3334; A61M 2205/6072; A61M 2230/04; A61M 2230/10; A61M 2230/42; A61M 2230/50; A61M 2205/3368

(57) ABSTRACT

The invention provides a device controlling and managing administration of infusion fluid for temperature regulation therapy, comprising at least one flow control unit for regulating flow rate of infusion fluid, at least one control unit for receiving input signals and providing output signals, wherein the control unit is configured to receive input signals that define infusion fluid to be administered or which is being administered, and store such information, and provide output signals to control electrolyte content of the infusion fluid based on said received input signals. A modified method for controlling and managing administration of infusion fluid for temperature regulation therapy is also provided.

### FIELD

The invention is directed to a device and methods for controlling and managing administration of infusion fluid, in particular for temperature regulation therapy, such as hypothermia treatment.

### INTRODUCTION

A condition in which the body's core temperature drops below that required for normal metabolism and body functions is usually called hypothermia. This is generally considered to be less than 35.0° C. (95.0° F.). Characteristic symptoms depend on the temperature. Targeted temperature management (TTM), previously known as therapeutic hypothermia, or protective hypothermia is an active treatment that tries to achieve and maintain a specific body temperature in a person for a specific duration of time in an effort to improve health outcomes. This is done in an attempt to reduce the risk of tissue injury from the lack of blood flow. Periods of poor blood flow may be due to cardiac arrest or the blockage of an artery by a clot, such as those that may occur during a stroke. Targeted temperature management improves survival and brain function following resuscitation from cardiac arrest. Evidence supports its use following certain types of cardiac arrest in which an individual does not regain consciousness. Targeted temperature management following traumatic brain injury has shown mixed results, with some studies showing benefits in survival and brain function, while others show no clear benefit. While associated with some complications, these are generally mild. Targeted temperature management can advantageously prevent brain injury by several methods including decreasing the brain's oxygen demand, reducing the proportion of neurotransmitters like glutamate, as well as reducing free radicals that might damage the brain. The lowering of body temperature may be accomplished by many means, including the use of cooling blankets, cooling helmets, cooling catheters, ice packs and ice water lavage.

Medical events where temperature management is beneficial for patient outcome fall primarily into conditions where the brain could suffer damage due to insufficient oxygen supply or elevated pressure on the brain tissue. This especially refers to conditions like: neonatal encephalopathy, cardiac arrest, ischemic stroke, traumatic brain or spinal cord injury without fever, and neurogenic fever following brain trauma.

Applicant's prior application WO2012143479, incorporated herein, provides a useful general description of an apparatus for temperature therapy. The disclosure generally relates to a device and method for controlling a temperature of a patient by an infusion of fluid. Said device comprises a supply of infusion fluid, a body temperature input adapted to receive the actual body temperature of the patient and an additional input adapted to receive at least one additional parameter representing the actual physiological state of the patient. Furthermore, the device comprises a control unit communicating with said body temperature input, and said additional input and at least one actuator which is in fluid communication with said supply and which controls the actual flow rate and/or actual temperature of the infusion fluid in accordance with at least one control signal of said control unit.

Document U.S. Pat. No. 7,896,834 B2, incorporated herein, discloses a pump system selectably controlling the temperature, flow rate, flow volume, and flow pressure of a fluid being infused into a patient's body. The apparatus comprises means for delivering a predetermined volume or halting device operation when an excessive volume has been infused.

Document U.S. Pat. No. 8,672,884 B2, incorporated herein, discloses methods for introducing fluids into a body cavity for hypothermic treatment. In one embodiment of the invention, at least one of the rate or volume of infusate is configured to increase a mean patient blood pressure. In another embodiment, the infusion parameter is at least one of a flow rate, a pressure, a total infused volume, an inflow duty cycle or a hypothermic solution temperature.

Infusing a solution into the blood stream of a patient will inherently affect the electrolytic balance of the blood, if the electrolytic profile of the infusion solution is not identical to that of the blood of the patient. The human body has functions to adjust for fluctuations in the concentration of cellular and extracellular electrolytes that result from dietary intake, metabolic activity and environmental stress, in order to keep these within narrow limits. It is especially critical that proper osmotic balance is maintained between intracellular and extracellular environment.

Temperature regulation therapy, such as therapeutic hypothermia treatment by infusion, frequently requires continuous infusion for one or more days and even for several days. It is an important therapeutic requirement that electrolytic balance is not jeopardized and that undue stress due to lowering of electrolyte concentration is minimized.

### SUMMARY

The present invention provides an improved device for controlling and managing administration of infusion fluid that takes account of the electrolytic balance requirements of the human body. Thus, the device of the invention regulates and monitors overall electrolytic component administered to a patient and provides recommendations for type of infusion fluid during continued temperature therapy and/or directly controls the type of infusion fluid being administered in order to optimize the electrolyte content of administered infusion fluid.

The term electrolyte refers to a substance whose components dissociate in solution into positively and negatively charged ions (cation and anion), and thus the term electrolytic component refers to any such component of an electrolyte.

Infusion fluid refers to any fluid administered intravenously to a patient. Substances that may be infused intravenously include volume expanders, blood-based products, blood substitutes, medications and nutrition. Infusion solutions can be broadly divided in crystalloid and colloid solutions. Crystalloids are aqueous solutions of mineral salts or other water-soluble small molecules that readily diffuse across semi-permeable membranes. Colloids contain larger colloid molecules, such as, but not limited to albumin, other blood proteins, gelatin, etc. that do not freely diffuse across a semi-permeable membrane (thus, blood is a colloid.) Accordingly, infusion solutions as defined herein include but are not limited to both crystalline solutions such as saline solutions or other type of conventional IV solutions (such as but not limited to those examples shown in Table 1), dissolved drugs or the like, and any type of colloid solution as well, administered to a patient via intravenous infusion.

The average daily fluid intake is about 2.5 L. The amount needed to replace losses from the urine and other sources is about 1 to 1.5 L/day in healthy adults. Other natural water losses are mostly regular losses from the skin and lungs (“insensible loss”), about 0.4 to 0.5 mL/kg/h on average or about 650 to 850 mL/day in a 70-kg adult. When a person has fever, another 50 to 75 mL/day may be lost for each degree C above normal temperature. GI losses are generally negligible, except when marked vomiting, diarrhea, or both occur. Sweat losses can be significant during environmental heat exposure or excessive exercise.

Water intake is regulated by thirst. Thirst is triggered by receptors in the anterolateral hypothalamus that respond to increased plasma osmolality (as little as 2%), or decreased body fluid volume. Rare hypothalamic dysfunction decreases capacity for thirst.

Water excretion by the kidneys is regulated primarily by vasopressin (ADH). Vasopressin is released by the posterior pituitary and results in increased water reabsorption in the distal nephron. Vasopressin release is stimulated by increased plasma osmolality, decreased blood volume, decreased blood pressure, and stress. Vasopressin release may be impaired by certain substances (eg, ethanol, phenytoin) and by central diabetes insipidus. Water intake decreases plasma osmolality. Low plasma osmolality inhibits vasopressin secretion, allowing the kidneys to produce dilute urine.

Water and sodium balance are closely interdependent. Total body water is about 60% of body weight in average weight in men and about 50% in women. Almost two thirds of total body water is in intracellular compartments (intracellular fluid or ICF), the other one third is extracellular (extracellular fluid, ECF), thereof about 25% is in the intravascular compartment; the other 75% being interstitial fluid. The major intracellular cation is potassium (K). The major extracellular cation is sodium (Na). Average concentrations of intracellular and extracellular cations are the following: K intracellular: 140 mEq/L, K extracellular 3.5 to 5 mEq/L, Na intracellular 12 mEq/L, Na extracellular 140 mEq/L.

Osmolarity is the term defining concentration of combined solutes in water, as amount of solute per L. In bodily fluids, this is similar to osmolality, which is the amount of solute per kg of solution. Water crosses cell membranes freely from areas of low solute concentration to areas of high solute concentration. Thus, osmolality tends to equalize across different body fluid compartments, resulting primarily from movement of water, not solutes. Solutes such as urea that freely diffuse across cell membranes have little or no effect on water shifts (little or no osmotic activity), whereas solutes that are restricted primarily to one fluid compartment, such as Na and K, have the greatest osmotic activity. Tonicity, or effective osmolality, reflects osmotic activity and determines the force drawing water across fluid compartments defined as the osmotic force.

Extracellular and intracellular fluid spaces are separated by cell membranes, with active sodium pumps, which ensure that sodium remains largely in the ECF. The cell also contains large anions such as protein and glycogen, which cannot escape and, therefore, draw in K ions to maintain electrical neutrality (Gibbs-Donnan equilibrium). These mechanisms ensure that Na⁺ and balancing anions, Cl⁻ and HCO₃⁻, are the key actors of ECF osmolality, and K⁺ has the corresponding function in the ICF.

The invention provides in one aspect a device for controlling and managing administration of infusion fluid(s) for temperature regulation therapy, wherein the device comprises at least one flow control unit for regulating flow rate of infusion fluid(s), and at least one control unit for receiving input signals and providing output signals. The control unit is configured to receive input signals that define infusion fluid to be administered or which is being administered, and to store such information, and provide output signals based on said received input signals. The output signals can in some embodiments affect the delivery of infusion fluid in order to deliver fluid with certain desired electrolyte content and/or alter the flow of fluid to alter the electrolyte content. Alternatively or additionally, the output signals comprise signals indicating recommendations for the therapy, at least including type of infusion fluid. Accordingly, the control unit can, in certain embodiments, provide signals to the flow control unit, to halt delivery or otherwise adjust delivery, e.g. change from one type of fluid to another, as further described herein.

The flow control unit refers to a device or arrangement that enables the device to actively maintain a certain flow rate, i.e. a pumping mechanism, that allows controlled, variable flow rate. As will be generally understood from the description provided herein, the flow control unit cooperates with the control unit, which provides the latter with control signals for maintaining or adjusting the flow rate. The pumping mechanism can comprise a pump of any kind available in the market, such as a peristaltic pump, piston pumps etc. The pump can be adapted to deliver the infusion fluid continuously and/or intermittently and/or sequentially, the latter preferably on the basis of pulses and intermediate pauses with volumes during the pulses of between 1 ml to 50 ml.

The device can, in some embodiments, accommodate more than one type of infusion fluid, such as typically by comprising or connecting to more than one infusion fluid container. Such embodiments allow different type of infusion fluid to be available and administered alternatively or in parallel. For example, if the control unit receives signals and determines based thereon that a change of infusion fluid is desirable, such as for altering the electrolyte content of the infusion fluid being administered, it can send a signal to the flow control unit to halt flow of infusion fluid A and initiate flow of infusion fluid B. (The terms infusion fluid ‘A’ and ‘B’ refer herein to any type of infusion fluid as further described herein, where A and B represent different infusion fluids. In certain embodiments, the unit can deliver simultaneously more than one type of infusion fluid, providing a desired mixture of fluids to the patient. Thus, as a non-limiting example, the control unit can provide a signal to reduce flow of fluid A by 50% and at the same time turn on flow of fluid B.

Such multi-fluid configurations as described herein above can e.g. be arranged with more than one pump, that is, one designated pump for each type of infusion fluid, followed by a joint leading to the delivery duct that brings the fluid to the patient.

The control unit generally comprises a processor and a memory for receiving and storing signal data, and for storing and executing programs for processing the received signals and controlling the flow control unit, and providing any suitable output signals and/or information as may be desired to implement. The control unit provides output signals that indicate at least recommendations for the therapy, most preferably recommendations as to type of infusion fluid to be used with respect to electrolytic content, and/or the output signals control the flow control unit, to increase or decrease (including halting) flow of an infusion fluid, as further described herein.

In one embodiment, the control unit is configured to receive input signals from at least one external computer system. This is particularly useful when used in hospitals using electronic patient journal systems that store and make available patient data such as biosignals (blood pressure, pulse, hemoglobin values, etc.), data from analysed patient samples, and data concerning administered therapy, including, but not limited to medicaments and fluids that have been or are being administered. It will be appreciated that the control unit is in some embodiments able to receive directly input from such at least one external computer system, with a suitable program interface to query the external system for the desired data. In other embodiments, the control unit prompts a user to feed the unit with desired data from such external computer system, manually, or by entering data files in suitable format (such as, but not limited to csv format or any other suitable format).

In some embodiments, the control unit is configured to receive input signals from at least one sensor that determines concentration of at least one electrolyte and provides a signal indicating said concentration. Such sensor can be arranged to determine the concentration of an electrolyte in an infusion fluid connected to the device, such as by arranging a special sampling duct delivering a quantity of fluid to the sensor, which can be but is not limited to at least one ion specific electrode, such as a sodium selective electrode, potassium selective electrode or chloride selective electrode. The sampling duct can be arranged in parallel with a delivery duct, such that fluid entering the sampling duct is consumed and discarded, or in-line with the delivery duct, analysing the fluid before it is delivered. Care must be taken to ensure that fluid administered to the patient remains sterile.

A sensor as described above and sampling duct can also be arranged on the patient, for sampling and analysing an electrolyte in the blood of the patient.

The term delivery duct as used in this context, refers to any conventional and useful delivery duct for an infusion fluid, such as a conventional infusion line and needle, well known to the skilled person.

In some embodiments, the control unit is configured to receive input signals that are entered by a user, who is typically a doctor or other caretaker. In such embodiments, the device comprises a user interface with a user information output such as a screen, for prompting the user for input signals to be entered, suitably via a touchpad screen or keyboard. Various arrangements are possible and within the scope of the invention. In some embodiments, the user is prompted at least whenever a fresh infusion bag is to be connected to the device and/or on regular time intervals. The input signals that are to be entered can, for example, be data defining which type of infusion fluid is connected, most suitably by choosing from a list stored in the memory of the device of typical conventional infusion fluids. Some common infusion fluids are defined below in a non-limiting list.

TABLE 1

| | Na⁺ | K⁺ | Ca⁺ | Mg⁺ | Cl⁻ | Lactate- | Acetate- | Osmolarity |
|---|---|---|---|---|---|---|---|---|
| | mmol/L | mmol/L | mmol/L | mmol/L | mmol/L | mmol/L | mmol/L | (mOsm/L) |
| NaCl | 154 | — | — | — | 154 | — | — | 308 |
| 0,9% | | | | | | | | |
| Ringer′s | 147 | 4.0 | 2.3 | — | 156 | — | — | 309 |
| soln | | | | | | | | |
| Ringer- | 125-134 | 4.0-5.4 | 0.9-2.0 | — | 106-117 | 25-31 | — | 262-293 |
| lactate | | | | | | | | |
| soln | | | | | | | | |
| Ringer- | 130 | 5.4 | 0.9 | 1.0 | 112 | — | 27 | 276 |
| acetate | | | | | | | | |
| soln. | | | | | | | | |

There are slight variations for the exact composition for some of the above mentioned solutions (such as Ringer's solution, Ringer's lactate soln, etc.) as supplied by different manufacturers, thus such terms should not be equated with one precise formulation.

In some embodiments, the device prompts the user for input such as every time an emptied or partially emptied bag is disconnected and every time a new bag is connected.

In other embodiments, the control unit is configured to receive input signals directly (without user input) from sensors, such as, but not limited to sensors for sensing vital signals or other patient signals (e.g. heart rate, blood pressure, EKG, EEG, temperature, breathing rhythm). In some embodiments, the control unit is able to receive a combination of input signals, both manually entered and received from sensors and/or external computers, systems, etc. Such signals can be used by the control unit for determining suitable infusion fluid. For example, such sensors may detect an adverse event, such as heart rate irregularities, which may indicate lack of potassium, then the control unit can respond by giving an output signal with instructions to change the infusion fluid to a fluid with higher potassium content. This can happen either such that the device will start administering potassium containing fluid instead of or in addition to non-potassium fluid, or by providing output signals to a user instructing to change infusion fluid.

Input signals that can be received from a sensor or system and/or entered in the device and which the device may prompt the user for may be selected from but are not limited to one or more of the following: signal indicating concentration of at least one electrolyte, signal indicating additional infusion fluid that the patient is being or is to be administered, signal indicating medication that the patient is being administered or has received, signal indicating medical condition of patient, signal indicating desired therapeutic body temperature of patient, and signal indicating blood status of patient. “Blood status” in this context may refer to any of various parameters describing status of blood, such as hemoglobin value, platelet count, etc. “Medical condition” in the context herein may refer to any vital signal such as but not limited to pulse, blood pressure, body temperature, or other relevant input parameter defining medical condition. In useful embodiments, the device is connected to one or more temperature sensors that provide the control unit with values indicating the body temperature of the patient.

The device may further comprise a unit to cool an infusion bag and/or keep it at desired temperature, and/or a sensor sensing the temperature of the infusion fluid in the infusion bag. Accordingly, cooled infusion fluid can be administered with this invention and the device described herein. The cooled infusion fluid preferably is delivered with a minimum temperature of 3.5° C., preferably 3.6° C., more preferably 3.7° C., more preferably 3.8° C., more preferably 3.9° C. and most preferably 4° C. and/or cooled infusion fluid is provided at a maximum temperature of 6° C., preferably 5.5° C., more preferably 5.0° C., more preferably 4.5° C., more preferably 4.25° C. and most preferably 4.0° C.

The adjustment device in one embodiment further comprises a fluid temperature unit for measuring and/or controlling the temperature of the medical fluid, and to deliver a respective temperature signal to the control unit.

The device of the present invention may suitably be arranged also with means to monitor and/or adjust volume being administered by controlling flow rate of IV fluid and monitoring fluid loss from the patient by suitable sensors, or prompting for relevant data to be entered representing fluid loss. Accordingly, in some embodiments of the invention, the device is configured to adjust the volume of one or more medical infusion fluids, the device comprising a at least one first determining unit adapted to measure and/or determine the volume of the medical infusion fluid flowing through a delivery duct and adapted to provide a respective first signal; at least one second determining unit adapted to measure and/or determine the volume and/or weight of at least one released body fluid and/or a physiological parameter and further adapted to provide a respective second signal; and at least one volume controlling unit adapted to control the flow of the medical infusion fluid through the delivery duct on the basis of the first and the second signals.

It is advantageous that the device be configured to minimize shivering of the patient. Shivering is a normal reflex reaction of the body to feeling cold, triggered to maintain homeostasis. Skeletal muscles begin to shake in small movements, creating warmth by expending energy. Thus, shivering will counteract the desired effect of hypothermia treatment in addition to being uncomfortable to the patient. In an embodiment of the invention, the control unit of the device is configured to receive input signals indicating levels of shivering and to provide output signals indicating one or more recommendation for therapy based on said received input signals, to counteract the shivering. In one such embodiment the control unit may react by forwarding an output signal to the flow control unit, signaling that the flow rate is to be altered (reduced), and/or the control unit may signal that the temperature of the infusion fluid is to be raised. In another embodiment, the control unit provides an output signal indicating a recommendation, or a signal to a drug delivery device, that the patient be administered an anti-shivering medication, such as but not limited to a medication selected from opiates, tramadol, magnesium sulfate, α2-agonists, physostigmine, doxapram, methylphenidate, and/or 5-HT3 antagonists. In other embodiments, output signal indicates a recommendation that surface temperature of the patient be affected, such as through the use of blankets, heating pads, or the like.

Intracranial pressure (ICP) is the pressure inside the skull and thus in the brain tissue and cerebrospinal fluid (CSF). Increased intracranial pressure (ICP) is one of the major causes of secondary brain ischemia that accompanies a variety of pathological conditions, most notably, traumatic brain injury (TBI), stroke, and intracranial hemorrhages. In some embodiments, the control unit of the device is further configured to receive input signals indicating intracranial pressure (ICP) and optionally blood pressure of a patient and to provide output signals indicating one or more recommendations for therapy based on said received input signals. Thus, the device can, in such embodiments, aid in the treatment of patients with elevated ICP. The input signals may be provided by a user, from an external computer system, or internally from a component of the device. The control unit may be configured to receive input signals indicating the level of intracranial pressure. ICP can be measured with invasive or noninvasive methods. Invasive methods normally require an insertion of an ICP sensor into the brain ventricle or parenchymal tissue. ICP can also be measured non-invasively. Several methods for noninvasive measuring of elevated ICP have been proposed: radiologic methods including computed tomography and magnetic resonance imaging, transcranial Doppler, electroencephalography power spectrum analysis, and the audiological and ophthalmological techniques. In one embodiment, the recommendation provided by the control unit comprises an instruction to administer ICP-reducing medication. As used herein, the term ICP-reducing medication is intended to mean any biologically active agent or drug or combination of agents or drugs that is administered to a patient for the purpose of reducing ICP. Any ICP-reducing agents can be used, such as agents commonly used in hyperosmolar therapy such as mannitol. In a preferred embodiment, the control unit is configured to provide output signals to a drug delivery device adapted to administer said ICP-reducing medication, where the delivery device is not part of the overall device.

A drug delivery device includes any means for containing and releasing a drug, wherein the drug is released to a subject. The term “drug delivery device” refers to any means for containing and releasing a drug, wherein the drug is released into a subject. The means for containing is not limited to containment in a walled vessel, but may be any type of containment device, including non-injectable devices (pumps etc.) and injectable devices, including a gel, a viscous or semi-solid material or even a liquid. Drug delivery devices may be inhaled, oral, transdermal, parenteral and suppository. Inhaled devices include gaseous, misting, emulsifying and nebulizing bronchial (including nasal) inhalers; oral includes mostly pills; whereas transdermal includes mostly patches. Parenteral includes injectable and non-injectable devices. Non-injectable devices may be “implants” or “non-injectable implants” and include e.g., pumps and solid biodegradable polymers. Injectable devices are split into bolus injections that are injected and dissipate, releasing a drug all at once, and depots, that remain discrete at the site of injection, releasing drug over time. Depots include e.g., oils, gels, liquid polymers and non-polymers, and microspheres. Many drug delivery devices are described in Encyclopedia of Controlled Drug Delivery (1999), Edith Mathiowitz (Ed.), John Wiley & Sons, Inc. The term “drug” as used herein, refers to any substance meant to alter animal physiology. The term “dosage form” refers to a drug plus a drug delivery device. The term “formulation” (or “drug formulation”) means any drug together with a pharmaceutically acceptable excipient or carrier such as a solvent such as water, phosphate buffered saline or other acceptable substance. A formulation may contain a drug and other active agents. It may also contain an excipient, solvent or buffer or stabilizing agent.

In another preferred embodiment, the control unit is configured to provide output signals to a drug delivery device which is part of the overall device. In other words, the device according to present invention comprises a drug delivery device and wherein the control unit is configured to provide output signals to said drug delivery device. Such device may be semi-automated or automated, such that when the ICP is above a certain given value, the control unit automatically provides an output signal to the drug delivery device adapted to deliver ICP-reducing medication to the patient without or with only minimal intervention of medical personnel.

In some embodiments, the device comprises means to detect automatically information on an infusion fluid bag, such as a barcode scanner or the like optical scanner, that reads information provided on suitable infusion bag, such as a barcode strip or other digitally readable label.

Based on the input information received, the device provides to the user recommendations to a user that includes information as to what type of infusion fluid is optimal.

As is understood from herein, the recommendations are determined by the control unit so as to maintain a desired and suitable electrolytic balance in the blood stream of the patient. For example, in a patient that is being cooled down by infusion to reduce negative effect on the brain after oxygen deprivation, but that has not lost any significant blood, may receive as initial IV fluid 0,9% NaCl solution, which is generally the most economical IV fluid to use for temperature therapy via IV infusion, and therefore recommended when other more elaborate multi-component fluids are not warranted. When the device has received information that the patient has received a certain number of IV bags, such as e.g. a total infusion volume of more than 2,5 L or more than 3 or 3.5 L, it may recommend to the user that another fluid be used, that includes further electrolytic components (e.g. Ringer's solution), to maintain an acceptable electrolytic balance in the blood stream. Similarly, if the device receives input signals indicating that the patient has low concentration of any particular vital electrolytic component, such as e.g. outside certain boundary values as determined critical, it may recommend as IV fluid a fluid comprising said electrolyte. Examples of such boundaries can be for example, if the patient is determined to have sodium blood levels of less than about 135 mmol/L, potassium levels of less than about 3,5 mmol/L, calcium blood concentration of less than about 1,15 mmol/L, and magnesium concentration of less than about 0,7 mmol/L. Other input parameters may as well determine recommended concentration electrolyte content of IV fluid, such as medical precondition of patient, duration of IV treatment, information on drugs administered to the patent, etc.

The device of the invention can advantageously be configured, so as to fit in a conventional hospital rack system, i.e. a bedside rack for containing one or more modular devices for patient care and/or monitoring. In such embodiments, the device is configured and designed as a modular unit to fit in such rack. The device can, in certain such embodiments, comprise more than a modular unit, for example when it is desired to actively cool the infusion fluid by keeping it in a cooled storage compartment while the fluid is administered. Such cooling compartment can be an add-on module.

### Specific Embodiments

The present invention will become more fully understood from the description before and particularly below and the accompanying drawings that are given by way of illustration only and show and/or exemplify preferred aspects thereof, and wherein

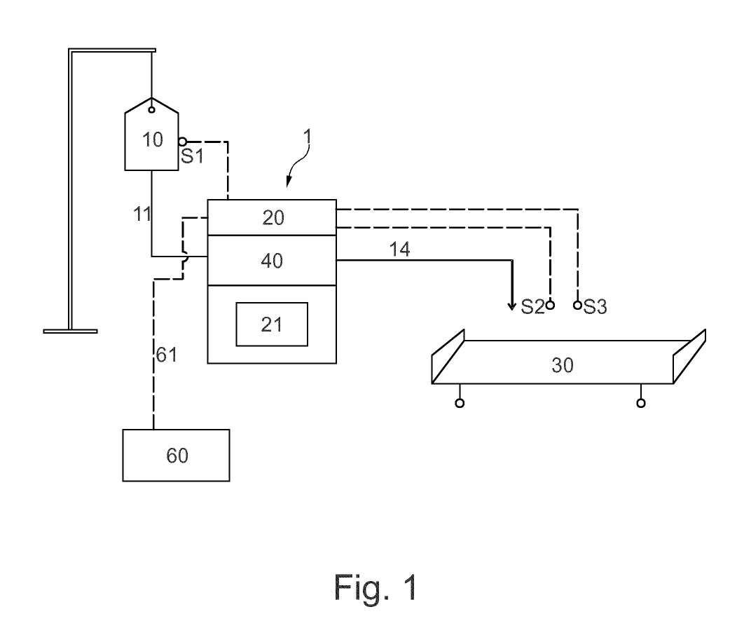

FIG. 1 illustrates a principal configuration of the device of the invention.

The device 1 as shown in FIG. 1 comprises a control unit 20, a flow control unit 40 and an input/output screen 21. A typical infusion fluid bag 10 is shown hung on a conventional supporting device. From the bag, a duct 11 provides infusion fluid through the flow control unit 40, which passes the infusion fluid onwards to a patient (not shown) through duct 14. An optional input line 61 from an external computer 60 is shown. Adjacent to the bag 10 is a barcode scanner/sensor S1 for detecting and registering the type of IV fluid bag, providing a signal to the control unit 20. Alternatively and optionally, input data concerning the type of IV fluid bag is input via the input screen 21.

Optional sensors S2, S3 are shown which can be connected to a patient, for feeding back signals to the control unit 20 with data indicating electrolyte blood concentration of the patient.

For further illustrative purpose, bed 30 is shown. Bed 30 can comprise a patient and symbolically refer to it.

The device further suitably will maintain a desired temperature of the IV fluid being administered (such as by containing the bag in a specialized compartment and/or by thermally insulating it), and preferably monitors the temperature of outgoing fluid and/or the IV bag being administered, and the body temperature of the patient. Such values, when monitored and registered by the device, are used to calculate and set a suitable flow rate. Thus, in some embodiments, the device comprises a temperature control unit that controls the temperature of IV fluid flowing from the device. Another or a plurality of reservoir(s) 10 can also be provided to provide either different infusion fluids as described above and/or the same infusion fluids for different purposes and/or with different temperatures. For such setup, a plurality of reservoir ducts 11 are provided (not shown).

In one demonstrative example of the use and utility of the device of the invention, and a specific non-limiting example of calculations and recommendations of the device, a patient in need of therapeutic hypothermia treatment is treated with the use of a device of the present invention. The device prompts the user to input measured values of electrolytes that include the sodium concentration in the patient's blood, and further prompts for input of a value corresponding to the body weight of the patient. The user inputs the value 110 mmol/L, which is the measured sodium blood concentration value for the particular patient, and 60 kg as patient body weight. The device in this embodiment considers a sodium value less than 135 mmol/L as a deficient value. The device calculates that the total sodium deficit amounts to 450 mmol (135-110 mmol/L×0,3×60 kg×1 L/kg=450 mmol) and outputs a recommendation that the patient be administered 0,9% saline soln, at least 2920 mL (450 mmol/L/154 mmol/L=2,92 L). In one configuration, the device further prompts the user to input the measured potassium concentration in the blood; if a value less than 3,5 mmol/L is added, the device recommends as IV solution Ringer's solution, containing 4,0 mmol/L potassium, and calculates the total volume which is suitable to overcome the potassium deficiency.

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components.

The present invention also covers the exact terms, features, values and ranges etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

The term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

It will be appreciated that variations to the foregoing embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” and the like, is merely intended to better illustrate the invention and does not indicate a limitation on the scope of the invention unless so claimed. Any steps described in the specification may be performed in any order or simultaneously, unless the context clearly indicates otherwise.

All of the features and/or steps disclosed in the specification can be combined in any combination, except for combinations where at least some of the features and/or steps are mutually exclusive. In particular, preferred features of the invention are applicable to all aspects of the invention and may be used in any combination.

1. A device for controlling and managing administration of infusion fluid for temperature regulation therapy, comprising:
at least one flow control unit for regulating flow rate of infusion fluid,
at least one control unit for receiving input signals and providing output signals,
wherein the control unit is configured to receive input signals that define infusion fluid to be administered or which is being administered, and store such information, and provide output signals to control electrolyte content of the infusion fluid based on said received input signals.

2. The device according to claim 1, wherein the output signals to control electrolyte content of the infusion fluid comprise output signals indicating recommendations for type of infusion fluid.

3. The device according to claim 1, wherein the control unit provides output signals to the flow control unit, for maintaining and/or adjusting flow rate of infusion fluid.

4. The device according to claim 1, wherein the control unit is configured to receive input signals from at least one external computer system.

5. The device according to claim 1, wherein the control unit is configured to receive input signals from at least one sensor that determines concentration of at least one electrolyte and provides a signal indicating said concentration.

6. The device according claim 1, wherein the control unit is configured to receive input signal entered by a user, and wherein the control unit prompts a user for input signals to be entered.

7. The device according to claim 1, wherein the input signals comprise signals indicating type of infusion fluid which is being infused or is to be infused as part of said therapy.

8. The device according to claim 1, wherein the input signals comprise signals selected from the group consisting of signals indicating concentration of at least one electrolyte, signals indicating additional infusion fluid that the patient is being administered, signals indicating medication that the patient is being administered, signals indicating medical condition of the patient, signals indicating desired therapeutic body temperature of the patient, and signals indicating blood status of the patient.

9. The device according to claim 1, wherein the control unit receives one or more input signals from one or more sensor.

10. The device according to claim 9, wherein the one or more signals are selected from signals indicating heart rate, blood pressure, temperature, breathing rhythm, EKG signals, and EEG signals.

11. The device according to claim 1, wherein the control unit receive input signals from a barcode scanner or corresponding sensor that automatically reads information on the infusion fluid bag to be administered.

12. The device according to claim 1, wherein the control unit provides an output signal comprising recommendations or instructions for type of infusion fluid to be administered.

13. The device according to claim 2, wherein the recommendations are determined by the control unit in order to maintain a desired electrolytic balance in the blood stream.

14. The device according to claim 1, which is configured to be connected to at least two containers of infusion fluid, wherein the flow control unit can deliver fluid from either one or both of said at least two containers.

15. The device according to claim 2, wherein the control unit further stores data indicating flow rate and amount of administered infusion fluid, and wherein said recommendations are further determined based on said data.

16. The device according to claim 12, wherein said instructions indicate if recommended subsequent infusion bag is of a type selected from
(a) isotonic sodium chloride solution;
(b) crystalloid solution comprising sodium, chloride, calcium and potassium;
(c) crystalloid solution comprising sodium, chloride, calcium, potassium, and one or both of acetate and lactate;
(d) solution comprising further electrolytic components; and
(e) blood solution or blood component solution.

17. The device according to claim 1, comprising a user interface which prompts the user for input information and outputs visual information indicating said output signals.

18. The device according to claim 2, wherein the control unit is further configured to receive input data indicating loss of patient fluids from the patient being treated, and wherein said recommendations are further determined based on said input data indicating loss of patient fluids.

19. The device according to claim 1, wherein the control unit is further configured to receive input data indicating body temperature of patient.

20. The device according to claim 1, wherein the control unit is further configured to receive input data indicating temperature of infusion fluid being administered and/or connected to the device.

21. The device according to claim 1, further comprising a temperature controlling unit, for controlling and maintaining desired temperature of infusion fluid.

22. A method for controlling and managing administration of infusion fluid for temperature regulation therapy, comprising administering an infusion fluid intravenously to a patient in need thereof, prompting a user for and/or receiving input signals that define the infusion fluid to a control unit, providing from the control unit output signals based on said received input signals indicating recommendations for the therapy, and further providing signals indicating suitable flow rate of the infusion fluid, and adjusting and/or maintaining the flow rate of the infusion fluid.

23. A method of treating a mammal comprising using the method according to claim 22.
